(19) United States
(12) Patent Application Publication
(10) Pub. No.: US 2004/0037431 A1
(43) Pub. Date: Feb. 26, 2004

(54) Active noise control system with modified spectral shaping path

(75) Inventor: Manish Vaishya, Auburn Hills, MI (US)

Correspondence Address: SIEMENS CORPORATION, INTELLECTUAL PROPERTY LAW DEPARTMENT, 170 WOOD AVENUE SOUTH, ISELIN, NJ, 08830, US

(73) Assignee: Siemens VDO Automotive, Inc.

(21) Appl. No.: 10/631,192

(22) Filed: Jul. 31, 2003

Related U.S. Application Data
(60) Provisional application No. 60/405,503, filed on Aug. 23, 2002.

(51) Int. Cl. A61F 11/06; G10K 11/16; H03B 29/00

(52) U.S. Cl. 381/71.8; 381/71.1

(57) ABSTRACT

An active noise control system (100) increases system stability by modifying a spectral shaping path (112) to prevent unbounded growth in the system error. In one embodiment, a model of the physical path (114) within the spectral shaping path (112) is given a positive bias, encouraging the model to overestimate the actual characteristics of the physical path (114). In another embodiment, the gain in the spectral shaping path (112) is normalized so that the gain decreases as the system output increases, placing an upper bound on the output signal. By modifying the model or the gain in the spectral shaping path (112), the invention improves system stability by limiting the destabilizing effects of modeling errors on the system.

### REFERENCE TO RELATED APPLICATIONS

The present invention claims the benefit of U.S. Provisional Patent Application No. 60/405,503, filed Aug. 23, 2002.

### TECHNICAL FIELD

The present invention is directed to active noise control for a vehicle, and more particularly to an active noise control model and system that controls noise in a vehicle engine.

### BACKGROUND OF THE INVENTION

Active noise control (ANC) systems are commonly used to control engine noise in vehicles. Generally, the ANC system outputs a generated sound having a characteristic that is an inverse of a characteristic of the sound generated by the engine. The characteristics of the generated sound is controlled by a control signal. When the generated sound and the engine sound combine together, they cancel each other out. Alternatively, the generated sound is designed to create a sound having a desired spectral content to modify the profile of the engine sound by cancelling and/or enhancing selected portions of the engine sound. The desired sound can change based on the sound actually generated by the engine, so the desired signal for generating the desired sound must be derived from the control signal itself.

The control signal traverses a physical path comprising combined transfer functions of components in the path, such as an amplifier, speaker, microphone, etc that may introduce their own physical effects into the desired signal. Because of these effects, the desired signal is filtered through a model of the physical path. The model can be represented as, for example, a finite impulse response (FIR) digital filter. This filter is applied when generating the desired signal to provide a desired spectral content in the output. Thus, the actual analog output of the ANC system is a difference between the desired sound and the engine sound.

Performance of the ANC system is highly dependent on the accuracy of the model, and any errors in the model result in an error in the system output. It is known that residual errors will always exist in the path model due to modeling inaccuracies and/or drift in the actual physical conditions of the ANC system.

In some situations, such as when the desired gain in the ANC system is high, the errors can be high enough to cause unbounded growth of the output, creating instability in the ANC system. More particularly, if the desired signal output from the model is lower than the ideal desired signal, the system will tend to become unstable. Because many models are generated using a least mean squares algorithm, which drives toward the ideal desired signal from a lower value, currently known systems tend to underestimate the model, leading toward possible system instability.

FIG. 1 is a graph illustrating an example of how errors in the model can cause system errors to increase toward infinity, particularly as the gain increases, as the model errors approach zero from the left side of the graph. More particularly, underestimating the model error may cause the output sound error to spike before reaching zero, causing system instability.

There is a desire for a model that can improve stability in an active noise control system.

### SUMMARY OF THE INVENTION

The present invention is directed to an active noise control system that increases system stability by modifying a spectral shaping path to prevent unbounded growth in the system error. In one embodiment, a model of the physical path within the spectral shaping path is given a positive bias, encouraging the model to overestimate the actual characteristics of the physical path. As a result, the error between the model and the actual physical path converges toward zero without encountering any singularities that may cause instability.

In another embodiment, the gain in the spectral shaping path is normalized so that the gain decreases as the system output increases, placing an upper bound on the output signal. This normalization drives the output to the correct value as well as reduces the system's sensitivity to modeling errors in the spectral shaping path. Normalizing the gain also ensures that the remainder of the algorithm used for noise control is unaffected, thereby preserving sound quality.

By modifying the model or the gain in the spectral shaping path, the invention improves system stability by limiting the destabilizing effects of modeling errors on the system.

### BRIEF DESCRIPTION OF THE DRAWINGS

FIG. 1 is a graph illustrating an example of an error in output sound versus a modeling error for various gain values;

FIG. 2 is a block diagram of an active noise control system incorporating one embodiment of the invention;

FIG. 3 is a graph illustrating two ways of introducing a positive bias in a model according to one embodiment of the invention;

FIGS. 4 through 6 are block diagrams illustrating examples of an active noise control system according to another embodiment of the invention.

### DETAILED DESCRIPTION OF THE EMBODIMENTS

Generally, the invention is directed to a method and system that controls engine sound via a digital model of a physical path in the active noise control (ANC) system. To improve system stability, one embodiment of the invention introduces a positive bias into the digital model by overestimating the physical path, introducing a positive bias in the model so that the output sound error will not spike as the model approaches a zero error. For example, with respect to FIG. 1, an overestimate in the model will cause error correction to move toward zero error from the right side of the graph as opposed to the left, allowing the model to reach zero error without encountering any system instability.

In another embodiment of the invention, a gain in the ANC system is normalized to place an upper bound on a gain in the ANC system to reduce the gain of the system as the output increases. This embodiment avoids changing the model itself, preserving the sound quality provided by the model while still improving the stability of the system. The normalization can be conducted using various different normalization equations.

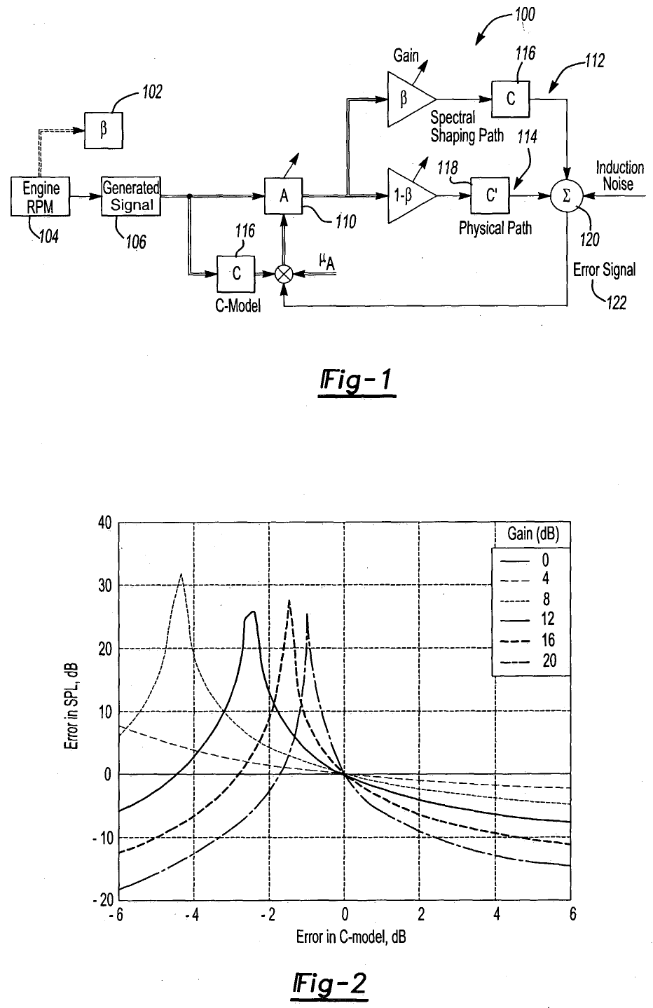

The invention will now be described in more detail below. FIG. 2 is a block diagram of an ANC system 100 that conducts noise cancellation and spectral shaping according to one embodiment of the invention. In this embodiment, β (block 102) represents a gain for a desired noise output from the system. In one embodiment, if β=0, the result is total cancellation of engine noise; the ANC system produces a generated noise having characteristics that are directly opposite the engine noise characteristics so that the generated noise and the produced noise cancel each other out completely. A β=1 leaves the engine noise completely unchanged. β values between 0 and 1 result in partial cancellation of the engine noise. β values greater than one enhance the engine noise without any cancellation.

Regardless of the value of β, the particular value of β is based on the engine speed (block 104) and the predetermined gain for each order in the spectrum of the engine noise. This is because the engine sound, and therefore the preferred engine sound, will change as the engine speed changes; an appropriate sound at a low engine speed, for example, would be different than an appropriate sound at a higher engine speed.

To ensure that the generated signal 106 will accurately produce the desired sound when mixed with the engine sound after being sent through a physical path in the ANC system, the generated signal 106 may be sent through a C-model 116 that represents the effect of various components in the physical path (e.g., speakers, microphones, electronic components, acoustic environment, etc.) on the generated sound. The specific model may vary depending on, for example, the sensitivity of the speaker and/or the microphone.

The generated signal is also sent through an adaptive filter 110 before being sent to a spectral shaping path 112 and a physical path 114. The adaptive filter 110 operation may be controlled by a convergence factor μ_{A}, which dictates how fast the ANC system 100 adapts to changes in the system 100. Tones in the generated sound that are to be enhanced are sent through the spectral shaping path 112, while tones to be controlled are sent through the physical path 114 to generate excitation to a speaker (not shown) in the system 100.

The spectral shaping path 112 includes a C-model 116 representing the ideal model of the physical path, while the physical path 114 includes a C′-model 118 representing a transfer function of the actual response of the physical path. Ideally, the difference between C and C′ models will be zero, indicating that the actual physical response of the system as represented by the C′-model 118 is identical to the ideal model of the physical path. However, any error between the C-model 116 and the C′-model 118 will remain in the system, unless the control system pauses for an update, in which case this error provides the feedback for correcting the C-model 116.

Once the C-model, C′-model, and the induction noise in the system are summed together (block 120), the resulting output of the summation 120 indicates the error 122 between the ideal and the actual response. This error 122 is sent back to the adaptive filter 110 so that the system 100 can adapt to the error and minimize the error signal.

Once the error is insignificantly small due to convergence between the physical path 114 and the spectral shaping path 112, the relationship between the induction noise, the gain β, and the total combined sound can be represented as follows:

Ñ−Ã(1−β)Ĉ=\tilde{P}ₒᵤₜₚₒₛₜ Equation 1

Ñ−Ã(1−β)Ĉ=βÃC Equation 2

where

Ã: adaptive filter matrix for FXLMS algorithm

Ñ: narrowband component of induction noise

Ĉ: transfer function of physical path

C: digital model of physical path

\tilde{P}ₒᵤₜₚₒₛₜ: net sound at orifice

β: desired gain in sound pressure

From the relationships described above, the net sound (i.e., the engine sound combined with the generated sound) can be described as follows:

$${\tilde{P}}_{output} = ( β \tilde{N} ) \frac{C}{( 1 - β ) \hat{C} + β C} \qquad Equation 3$$

where BN is the ideal sound output. The net error in the sound can be described as:

$$Δ E = \frac{{\tilde{P}}_{output}}{β \tilde{N}} = \frac{1 + Δ C / \hat{C}}{1 + β Δ C / \hat{C}} \qquad Equation 4$$

where ΔC=C−C′. In a perfect model, AC will equal zero because the C-model 116 will match the actual physical path represented by C′-model 118, and ΔE will be equal to 1 by cancelling out any effects of β on the final error ΔE. As can be seen in Equation 4 and FIG. 1, ΔE will go to infinity as 1+βΔC/C) approaches zero, which would occur if ΔC is negative. Because ΔC will be negative only if the C-model underestimates the actual physical path C′-model, overestimating the C-model will prevent ΔC from becoming a negative value, ensuring that the system will always be stable as it approaches zero error.

Although it theoretically may be difficult to generate an overestimate of the actual physical path without knowing what the transfer function of the C′-model will look like, constructing the ideal C-model by starting with a large overestimate solves this problem. A large overestimate in the C-model may result in a large error AC at first, but the feedback provided by the error signal 112 will cause the ideal C-model 116 to converge quickly to the actual physical path represented by the C′-model 118 without ever causing the C-model error to go negative and cause instability. With reference to FIG. 1, overestimating the C-model 116 will cause ΔC to approach zero from the right side of the graph and not encounter any singularities where ΔE goes toward infinity even at high gains.

FIG. 3 illustrates one way of introducing a positive bias in the C-model 116 (e.g., ensure that ΔC is always greater than 0). In one embodiment, a predictive model may be used to estimate C-model value where curve fitting is used to estimate an asymptotic final value. This value is then amplified by the bias amount, which is usually a fraction of the estimated asymptotic final value. From this, the C-model will converge toward the actual physical C′-model from the positive direction rather than the negative direction. Alternatively, a higher order characteristic may be incorporated into adaptive filter equation so that the C-model will overshoot. Other methods will be apparent to those of ordinary skill in the art and can be incorporated into the ANC system. Regardless of the specific method used to introduce the positive bias in the C-model, the least mean squares algorithm used to converge the C-model toward the actual physical path will drive the error toward zero.

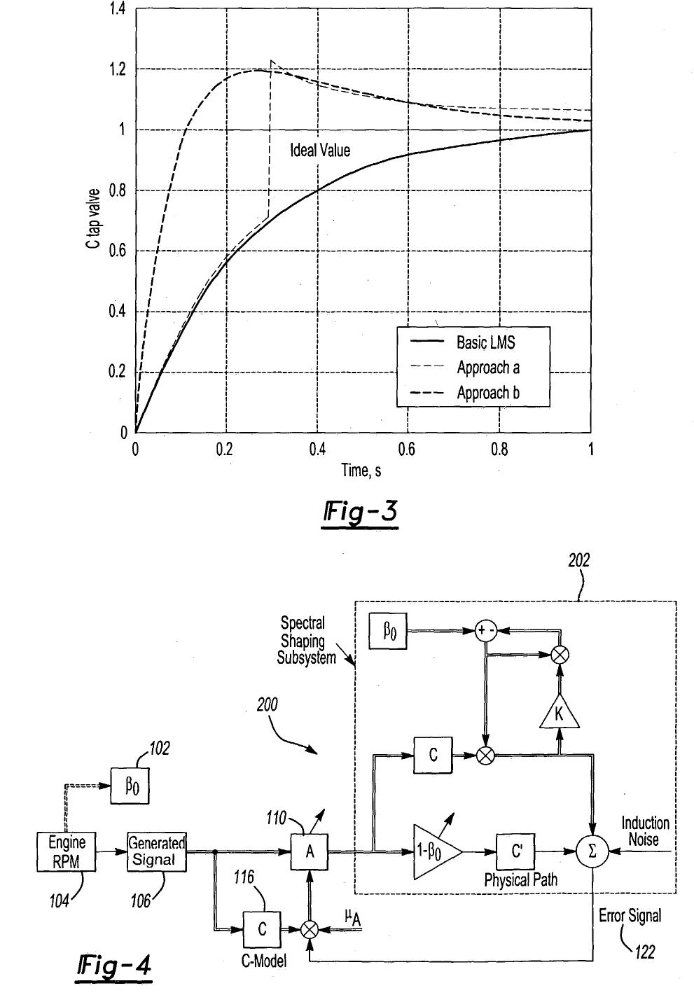

FIGS. 4, 5 and 6 illustrate alternative ways of improving the stability of an ANC system. In these embodiments, the spectral shaping path 112 is modified to normalize the gain value β so that the system 100 is less sensitive to modeling errors in the C-model 116. In one embodiment, the gain β is normalized to reduce the gain as the system output increases to drive the output toward the correct value. Normalizing the gain leaves the remainder of any control algorithms in the system 100 unaffected.

In one embodiment, the normalization is conducted without introducing any significant offset in the system, as would be the case in simple output limiting or power leakage techniques, to preserve consistent sound quality. Further, the normalization should be non-dimensionalized with respect to the magnitude of the C-model 116 so that changes in the physical path 114 will have a minimal effect on system performance. Various normalization equations are described below for illustrative purposes only; those of skill in the art will be able to determine which equations are most appropriate for a given sound level and characteristic.

In the examples below, the output of the ANC system treats the gain values β in the physical path and the spectral shaping path as independent values β₁ and β₂, respectively. The output of the ANC system incorporating normalization can then be expressed as:

$${\tilde{P}}_{output} = ( {β}_{2} \tilde{N} ) \frac{C}{( 1 - {β}_{1} ) \hat{C} + {β}_{2} C} \qquad Equation 5$$

From this equation, either gain value β₁ or β₂ can be normalized with respect to either the ideal ANC system output or the actual ANC system output, and either gain value β₁ or β₂ can be assumed to be an ideal gain β₀ for normalization purposes.

FIG. 4 is a block diagram illustrating an ANC system 200 according to one embodiment of the invention incorporating normalization. This system 200 is similar to the system shown in FIG. 3 except that the spectral shaping path 116 and physical path 118 have been modified to form a spectral shaping subsystem 202 incorporating normalization of the gain β. In this embodiment, the value of β₁ in Equation 5 is assumed to be the ideal gain (β₁=β₀) while β₂ is normalized with respect to the actual system output. As a result, the normalized gain β₂ can be written as:

$$\begin{matrix}{β}_{2} = \frac{{β}_{0}}{1 + K {\tilde{P}}_{output}} ; & & & {β}_{1} = {β}_{0}\end{matrix} \qquad Equation 6$$

where K is a normalization coefficient, which can be determined from acceptable limits of residual error. As can be seen in Equation 6, the gain β₂ in the spectral shaping path decreases as the output power Poutput increases, thereby limiting uncontrolled growth of the output.

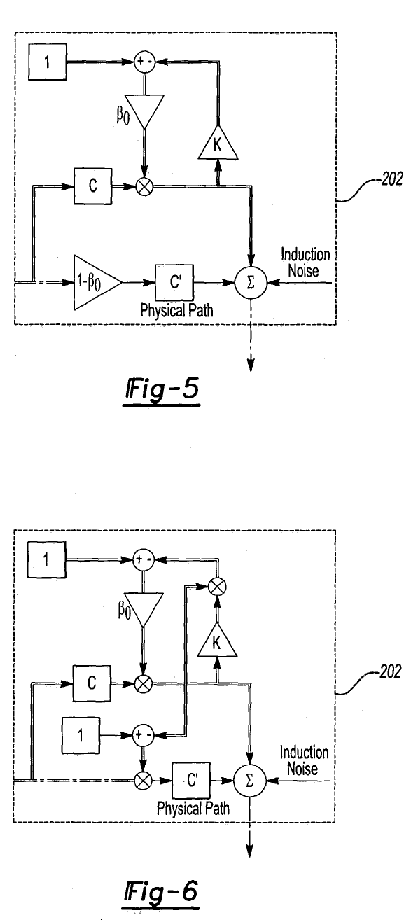

FIG. 5 illustrates a variation of the spectral shaping subsystem 202 in FIG. 4. In this variation, the gain value β₂ in the spectral shaping path is normalized with respect to an ideal (as opposed to an actual) system output. The gain β₁ in the physical path is assumed to be the ideal gain β₀, generating the following equation:

$${β}_{2} = \frac{{β}_{0}}{1 + K {\tilde{P}}_{ideal}} ; {β}_{1} = {β}_{0} ⇒ {β}_{2} = {β}_{0} ( 1 - K {\tilde{P}}_{output} ) \qquad Equation 7$$

FIG. 6 illustrates yet another variation of the spectral shaping subsystem 202. In this variation, the gain β₂ in the spectral shaping path is normalized with respect to the actual system output as well as the ideal gain value β₀. In this variation, the gain in the physical path β₁ and the gain in the spectral shaping path β₂ are set to be equal to each other, generating the following equation in the spectral shaping path:

$$\begin{matrix}{β}_{2} = \frac{{β}_{0}}{1 + K {β}_{0} {\tilde{P}}_{output}} ; & & & {β}_{1} = {β}_{0}\end{matrix} \qquad Equation 8$$

These methods illustrate some of the normalizing techniques that can be applied. The selection of specific method will usually be based on the trade-off between stability and accuracy, and also on the specific zone of operation within the scope of FIG. 1.

By modifying the spectral shaping path either by introducing a positive bias in the C-model or normalizing the gain in the spectral shaping path, the invention improves the stability of the ANC system by preventing the error in the output from increasing to uncontrolled levels even with the gain in the system is high.

It should be understood that various alternatives to the embodiments of the invention described herein may be employed in practicing the invention. It is intended that the following claims define the scope of the invention and that the method and apparatus within the scope of these claims and their equivalents be covered thereby.

What is claimed is:

1. A method of controlling an active noise control system, comprising:
determining an ideal model of a physical path of the active noise control system, wherein the ideal model overestimates an actual response of the physical path;
generating an actual response using the ideal model;
calculating a difference between an ideal response and the actual response to obtain an error signal;
adjusting the ideal model based on the error signal.

2. The method of claim 1, wherein the overestimate in the ideal model causes the error signal to always be a positive value.

3. The method of claim 1, wherein the adjusting step adjusts the ideal model toward the actual response to reduce the error signal.

4. The method of claim 1, further comprising controlling a rate at which the adjusting step is conducted according to a conversion factor.

5. The method of claim 1, wherein the overestimate in the physical path is obtained by a predictive model.

6. The method of claim 1, wherein the overestimate in the physical path is obtained by incorporating a higher order characteristic in the filter update equation during the adjusting step.

7. A method of controlling an active noise control system, comprising:
defining a first gain in a physical path and a second gain in a spectral shaping path;
normalizing the second gain based on a system output value;
generating an actual response using an ideal model and the normalized second gain;
calculating a difference between an ideal response and the actual response to obtain an error signal;
adjusting the system model based on the error signal.

8. The method of claim 7, wherein the system output value is the actual response.

9. The method of claim 8, wherein the second gain is calculated by dividing an ideal gain by a value based on the actual response.

10. The method of claim 9, wherein the ideal gain is equal to the first gain.

11. The method of claim 8, wherein the second gain is calculated by dividing an ideal gain by a value based on the actual response and the ideal gain.

12. The method of claim 11, wherein the ideal gain is equal to the first gain.

13. The method of claim 7, wherein the system output value is the ideal response.

14. The method of claim 13, wherein the second gain is calculated by dividing an ideal gain by a value based on the ideal response.

15. An active noise control system, comprising:
a sound generator that outputs a generated sound based on an engine operating characteristic;
a physical path through which the generated sound travels, the physical path having a first gain;
a spectral shaping path having an ideal model of the physical path and a second gain, wherein the generated sound is controlled by the ideal model and the second gain to generate an actual response;
a controller that calculates a difference between an ideal response of the active noise control system and the actual response to obtain an error signal and adjusts the system model based on the error signal.

16. The system of claim 15, wherein the ideal model initially overestimates the actual response.

17. The system of claim 15, further comprising a spectral shaping subsystem that normalizes the second gain based on a system output value, wherein the actual response is generated using the ideal model and the normalized second gain.

18. The system of claim 17, wherein the system output value is the actual response.

19. The system of claim 18, wherein the second gain is calculated by dividing the first gain by a value based on the actual response.

20. The system of claim 18, wherein the second gain is calculated by dividing the first gain by a value based on the actual response and the first gain.

21. The system of claim 17, wherein the system output value is the ideal response, and wherein the second gain is calculated by dividing an ideal gain by a value based on the ideal response.
